(19) United States
(12) Patent Application Publication
(10) Pub. No.: US 2023/0094267 A1
(43) Pub. Date: Mar. 30, 2023

(54) METHOD FOR NATURAL KILLER CELL EXPANSION

(71) Applicant: ACADEMIA SINICA, Taipei City (TW)

(72) Inventors: Che-Ming Jack HU, Taipei City (TW); Shih-Yu CHEN, Taipei City (TW); Yi-Fu WANG, Taipei City (TW); Wan-Chen HSIEH, Taipei City (TW); Yi-Shiuan TZENG, Taipei City (TW); Ya-Ting LU, Taipei City (TW); Jung-Chen LIN, Taipei City (TW); Chung-Yao HSU, Taipei City (TW)

(73) Assignee: ACADEMIA SINICA, Taipei City (TW)

(21) Appl. No.: 17/908,585

(22) PCT Filed: Mar. 2, 2021
(86) PCT No.: PCT/US2021/020430

§ 371 Date: Sep. 1, 2022

Related U.S. Application Data
(60) Provisional application No. 62/984,060, filed on Mar. 2, 2020.

(51) Int. Cl. C12N 5/0783; C07K 14/705; C07K 14/54

(52) U.S. Cl. CPC C12N 5/0646; C07K 14/70575; C07K 14/5443; C12N 2501/2302; C12N 2501/2321; C12N 2501/734; C12N 2529/10

(57) ABSTRACT

A method of expanding natural killer cells, comprising: providing a population of internally gelated cells, each of which includes a gelated interior and a fluid cell membrane that contains one or more membrane-bound proteins each or collectively are capable of stimulating expansion of natural killer (NK) cells; and culturing a population of cells containing NK cells, which are capable of responding to the one or more membrane-bound proteins, with the population of internally gelated cells under conditions that allow expansion of NK cells.

### CROSS REFERENCE

This application claims priority to U.S. Provisional Application No. 62/984,060, filed on Mar. 2, 2020, the entire content of which is incorporated by reference herein.

### BACKGROUND

Natural killer (NK) cells, comprising 10-15% of peripheral blood lymphocytes, play an important role in immune surveillance due to their innate ability to kill cancer and virally infected cells without prior sensitization. See, Abel et al., Front. Immunol. 9, 1869 (2018); Cerwenka and Lanier, Nat. Rev. Immunol. 16, 112-123 (2016); Adams et al. J. Immunol. 197, 2963-2970 (2016); and Chiossone et al., Nat. Rev. Immunol. 18, 671-688 (2018). NK cells are identified by their surface expression of CD56 and absence of the T cell marker CD3. A subset of NK cells expresses the FcγRIII protein, CD16, which enhances NK cell cytotoxic function by aiding antibody-dependent cellular cytotoxicity (ADCC). See, Cerwenka and Lanier, Nat. Rev. Immunol. 16, 112-123 (2016); Adams et al. J. Immunol. 197, 2963-2970 (2016); and Freud et al., Immunity 47, 820-833 (2017).

NK cell function is largely controlled by families of cell surface activating and inhibitory receptors. Activation signals are transduced by activating receptors such as NKG2D that recognize ligands including the stress-induced protein MICA. Inhibitory receptors recognize molecules, such as MHC class I, that are universally expressed on normal cells and frequently downregulated in cancer cells. By monitoring the balance between activating and inhibitory receptors, NK cells are able to recognize and kill stressed cells such as infected cells or cancer cells. See, Cerwenka and Lanier, Nat. Rev. Immunol. 16, 112-123 (2016); Chiossone et al., Nat. Rev. Immunol. 18, 671-688 (2018); and Fujisaki et al., Cancer Res. 69, 4010-4017 (2009).

NK cells have been implicated in tumor immunosurveillance based on numerous mouse models and human studies. Given their strong anti-tumor activities, adoptive cell therapies using NK cells are attractive therapeutic approaches against cancer. See, Cerwenka and Lanier, Nat. Rev. Immunol. 16, 112-123 (2016); Fujisaki et al., Cancer Res. 69, 4010-4017 (2009); Cheung et al., Nat. Rev. Cancer 13, 397-411 (2013); and Brodeur et al., Nat. Rev. Cancer 3, 203-216 (2003).

Therefore, there is a need to establish an expansion system to obtain large number of highly potent NK cells for clinical applications.

### SUMMARY

The details of one or more embodiments are set forth in the accompanying drawing and the description below. Other features, objects, and advantages of the embodiments will be apparent from the description and drawing, and from the claims.

In one aspect, a method of expanding natural killer cells is described herein. The method includes providing a population of internally gelated cells, each of which includes a gelated interior and a fluid cell membrane that contains one or more membrane-bound proteins each or collectively are capable of stimulating expansion of natural killer (NK) cells; and culturing a population of cells containing NK cells, which are capable of responding to the one or more membrane-bound proteins, with the population of internally gelated cells under conditions that allow expansion of NK cells.

In some embodiments, the population of cells is selected from the group consisting of peripheral blood mononuclear cells (PBMCs), enriched NK cells, iPSC-derived NK cells, embryonic stem cell-derived NK cells, tissue resident NK cells, splenocytes, cord blood cells, and hematopoietic stem cell-derived NK cells.

In some embodiments, the one or more membrane-bound proteins are selected from the group consisting of 41BBL, IL-15, IL-21, B7-H6, BAT3, HLA-DP, HLA-E, HLA-C2, HLA-A, HLA-C, HLA-G, HLA-F, HLA-C, MICA/MICB, ULBP-1, ULBP-2, ULBP-3, ULBP-4, ULBP-5, ULBP-6, AICL, CD48, NTB-A, 2B4, CD2, CD58, CD11a, ICAM1, CRACC, OX40L, CD137L, Nectin-1, Nectin-2, Nectin-3, Nectin-4, necl-1, necl-2, necl-3, necl-4, necl-5, PCNA, AICL, IgG, CD27L, CD72, CEACAM-1, CEACAM-5, OCIL, N-Cadherin, E-Cadherin, R-Cadherin, sialic acid, IL-1, IL-2, IL-4, IL-7, IL-9, IL-12, IL-18, IL-27, IL-33, IL-6, IL-11, CNTF, LIF, OSM, CT-1, CLC, IFN-a, INF-b, CCL-5, and an agonist of TLR-1, TLR-2, TLR-3, TLR-5, TLR-6, TLR-9, NOD-1, NOD-2, NOD-3, or NLRP3. For example, the one or more membrane-bound proteins can include 41BBL and IL-15.

In some embodiments, the culturing step is performed in the presence of IL-21 or IL-2.

In some embodiments, the ratio of the number NK cells to the number of gelated cells is 1:0.5-20 (e.g., 1:1, 1:2, 1:3, 1:4, 1:5, 1:6, 1:7, 1:8, 1:9, 1:10, 1:12, 1:15, or 1:20).

In some embodiments, the population of internally gelated cells is generated by a procedure including: providing a population of antigen presenting cells that express the one or more membrane-bound proteins; suspending the population of antigen presenting cells in phenol-red free DMEM containing a protease inhibitor cocktail to generate a first cell suspension; adding a gelation solution to the first cell suspension to generate a second cell suspension, wherein the gelation solution is capable of increasing membrane permeability of the antigen presenting cells, and contains a photo-reactive crosslinker and an optional photo-initiator; incubating the second cell suspension at room temperature for a period of time sufficient to allow the photo-reactive crosslinker and the optional photo-initiator to enter the antigen presenting cells; centrifuging the second cell suspension to generate a cell pellet: resuspending the cell pellet in phenol-red free DMEM to generate a third cell suspension; applying a light to the third cell suspension for a period of time sufficient to allow cross-linking of the photo-reactive crosslinker, whereby the population of internally gelated cells is generated; and collecting and washing the population of internally gelated cells.

In some embodiments, the gelation solution is prepared such that the second cell suspension has an osmotic concentration of 320 mOsmol to 290 mOsmol, greater than 320 mOsmol, or lower than 290 mOsmol.

In some embodiments, the gelation solution contains dimethyl sulfoxide (DMSO) such that the second cell suspension contains 0.1 to 5 wt % of DMSO.

In some embodiments, the concentration of the photo-reactive crosslinker in the second cell suspension is 5 wt % to 50 wt %.

In some embodiments, the photo-reactive crosslinker is poly(ethylene glycol)-diacrylate (PEG-DA), the photo-initiator is 2-hydroxy-4′-(2-hydroxyethoxy)-2-methylpropiophenone, and the light is 365 nm blue light. For example, the 2-hydroxy-4′-2(-hydroxyethoxy)-2-methylpropiophenone can range from 0.01 to 1 wt % in the gelation solution, and the PEG-DA has an average molecular weight between 200 Da to 5000 Da ranging from 2 to 80 wt % in the gelation solution.

In some embodiments, the gelation solution is prepared by dissolving 2-hydroxy-4′-(2-hydroxyethoxy)-2-methylpropiophenone in DMSO to create a solution and mixing the solution with PEG-DA having an average molecular weight of 700 Da.

In some embodiments, the concentration of PEG-DA in the second cell suspension is 10 wt % to 40 wt %.

In some embodiments, the population of antigen presenting cells are artificial antigen presenting cells. For example, the artificial antigen presenting cells can be or engineered from K562 cells, PBMC, EBV transformed LCL, 721.221 cells, 8866 cells, Jurkat cells, Jurkat/KL-1 cells, U937 cells, BJAB cells, NB4 cells, 293T cells, MCF7 cells, Jeg3 cells, Hela cells, A549 cells, 1106mel cells, or CEM cells.

In some embodiment, the method further includes administering the expanded NK cells to a subject in need thereof, e.g., a subject having a cancer, an infection, an autoimmune disorder, NK cell-deficient condition, or unwanted cells.

In one aspect, described herein is a method of treating a disease, including administering to a subject in need thereof the expanded NK cells generated by the gelated cells described herein. In some embodiments, the disease is a cancer, an infection, an autoimmune disorder or NK cell-deficient condition, such as classical NK deficiencies and functional NK deficiencies, or a condition of having unwanted cells.

In another aspect, described herein is a method of generating a population of internally gelated cells. The method includes: providing a population of precursor cells that express one or more membrane-bound proteins; suspending the population of precursor cells in phenol-red free DMEM containing a protease inhibitor cocktail to generate a first cell suspension; adding a gelation solution to the first cell suspension to generate a second cell suspension, wherein the gelation solution is capable of increasing membrane permeability of the precursor cells, and contains a photo-reactive crosslinker and an optional photo-initiator; incubating the second cell suspension at room temperature for a period of time sufficient to allow the photo-reactive crosslinker and the optional photo-initiator to enter the precursor cells; centrifuging the second cell suspension to generate a cell pellet; resuspending the cell pellet in phenol-red free DMEM to generate a third cell suspension; applying a light to the third cell suspension for a period of time sufficient to allow cross-linking of the photo-reactive crosslinker, whereby the population of internally gelated cells is generated; and collecting and washing the population of internally gelated cells; wherein the internally gelated cells each include a gelated interior and a fluid cell membrane that contains the one or more membrane-bound proteins.

In some embodiments, the gelation solution is prepared such that the second cell suspension has an osmotic concentration of 320 mOsmol to 290 mOsmol, greater than 320 mOsmol, or lower than 290 mOsmol.

In some embodiments, the gelation solution contains DMSO such that the second cell suspension contains 0.1 to 5 wt % of DMSO.

In some embodiments, the concentration of the photo-reactive crosslinker in the second cell suspension is 5 wt % to 50 wt %.

In some embodiments, the photo-reactive crosslinker is poly(ethylene glycol)-diacrylate (PEG-DA), the photo-initiator is 2-hydroxy-4′-(2-hydroxyethoxy)-2-methylpropiophenone, and the light is 365 nm blue light. For example, the 2-hydroxy-4′-2(-hydroxyethoxy)-2-methylpropiophenone can range from 0.01 to 1 wt % in the gelation solution, and the PEG-DA can have an average molecular weight between 200 Da to 5000 Da ranging from 2 to 80 wt % in the gelation solution.

In some embodiments, the gelation solution is prepared by dissolving 2-hydroxy-4′-(2-hydroxyethoxy)-2-methylpropiophenone in DMSO to create a solution and mixing the solution with PEG-DA having an average molecular weight of 700 Da.

In some embodiments, the concentration of PEG-DA in the second cell suspension is 10 Wt % to 40 wt %.

In some embodiments, the population of precursor cells are artificial antigen presenting cells. For example, the artificial antigen presenting cells can be or engineered from K562 cells, PBMC, EBV transformed LCL, 721.221 cells, 8866 cells, Jurkat cells, Jurkat/KL-1 cells, U937 cells, BJAB cells, NB4 cells, 293T cells, MCF7 cells, Jeg3 cells, Hela cells, A549 cells, 1106mel cells, or CEM cells.

In some embodiments, the one or more membrane-bound proteins each or collectively are capable of stimulating expansion of natural killer (NK) cells.

In some embodiments, the one or more membrane-bound proteins are selected from the group consisting of 41BBL, IL-15, IL-21, B7-H6, BAT3, HLA-DP, HLA-E, HLA-C2, HLA-A, HLA-C, HLA-G, HLA-F, HLA-C, MICA/MICB, ULBP-1, ULBP-2, ULBP-3, ULBP-4, ULBP-5, ULBP-6, AICL, CD48, NTB-A, 2B4, CD2, CD58, CD11a, ICAM1, CRACC, OX40L, CD137L, Nectin-1, Nectin-2, Nectin-3, Nectin-4, necl-1, necl-2, necl-3, necl-4, necl-5, PCNA, AICL, IgG, CD27L, CD72, CEACAM-1, CEACAM-5, OCIL, N-Cadherin, E-Cadherin, R-Cadherin, sialic acid, IL-1, IL-2, IL-4, IL-7, IL-9, IL-12, IL-18, IL-27, IL-33, IL-6, IL-11, CNTF, LIF, OSM, CT-1, CLC, IFN-a, INF-b, CCL-5, and an agonist of TLR-1, TLR-2, TLR-3, TLR-5, TLR-6, TLR-9, NOD-1, NOD-2, NOD-3, or NLRP3.

In yet another aspect, described herein is a population of internally gelated cells generated by the method described herein, wherein each cell comprises a gelated interior and a fluid cell membrane that contains the one or more membrane-bound proteins.

In one aspect, described herein is a composition comprising the population of internally gelated cells.

In another aspect, provided herein is a method of inducing an immune response in a subject, comprising administering the composition to the subject.

### BRIEF DESCRIPTION OF DRAWINGS

FIG. 1 is a schematic illustration of an exemplary process of preparing gelated cells.

FIG. 2 is a set of images showing live K562 cells and gelated K562 cells. Live K562 cells (left) and gelated K562 cells (right) were visually affirmed to possess similar cellular morphology prior to further experimentation.

FIG. 3 is a set of graphs showing NK cell expansion by a novel feeder cell system. PBMC were co-cultured with irradiated K526-41BBL-mb15 cells (GM) and gelated cells prepared from K526-41BBL-mb15 cells (GC) with or without IL-21 (100 ng/ml). Cell population (A), NK cell numbers (B), and expansion fold (C) were determined on day 0, 7, and 14 (n=3). Error bars represent the mean±SD.

FIG. 4 is a set of graphs showing that NK cells exhibited increased cytotoxicity after expansion by GC. PBMC were co-cultured with GM or GC with or without IL-21 and with (B) or without (A) anti-CD137 antibody. The cytotoxicity of expanded NK cells was assessed via killing assay. E:T ratios were 1:1, 0.5:1, and 0.25:1 (n=2). Error bars represent the mean±SD.

FIG. 5 is a set of graphs showing that GC increased the cytolytic activity of NK cells. (A) Total cell number (left), NK population (middle), and NK cell number (right) were determined for GM and GC expansion systems. (B) The expansion folds for GM-expanded NK cells and GC-expanded NK cells were determined after expansion for 7 days. (C) The cytolytic activity of NK cells were determined after expansion for 7 days. (D) The GM and GC populations in the system during NK expansion were determined.

FIG. 6 is a set of graphs showing optimization of conditions of GC expansion system. (A) NK cells were expanded by different GC with different stiffness. The total NK cell number was determined after expansion for 7 days. (B) The cytolytic activity for NK cells expanded from GC was determined after expansion for 7 days.

FIG. 7 is a set of graphs showing optimization of expansion conditions NK cells enriched from PBMC. PBMC-enriched NK cells were expanded using different ratio of cells (NK:feeder=1:10, 1:5, 1:2, and 1:0.5) with 10 IU/mL IL-12 (A-C) or 100 IU/mL IL-2 (D-F). (A)(D) The total cell number (left), NK population (middle), and NK cell number (right) for NK cells expanded under different cell ratios were assessed on day 0 and day 7. (B)(E) The fold of expansion under different cell ratios was determined after expansion for 7 days. (C)(F) The cytolytic activity of NK cells expanded under different cell ratios was determined after expansion for 7 days.

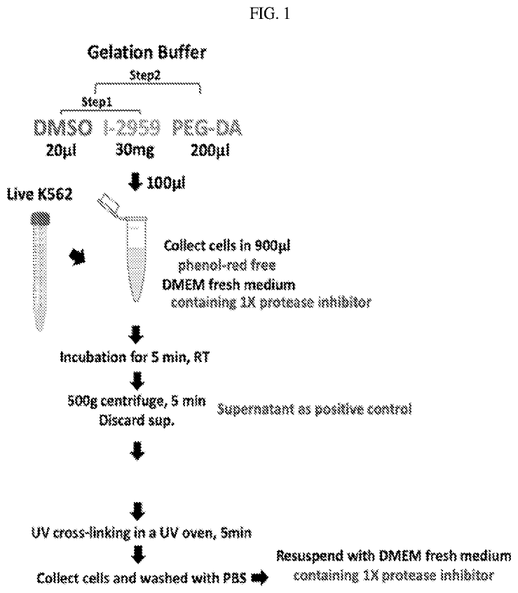

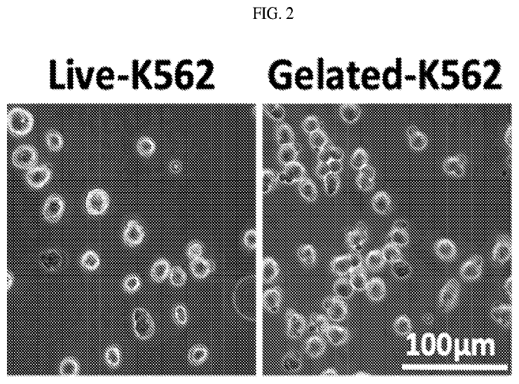

### DETAILED DESCRIPTION

It was demonstrated that the intracellular hydrogelation technique could preserve cell plasma membrane integrity while acquiring extraordinary stability. See, Lin et al., Nat. Commun. 10, 1057 (2019). By preparing artificial antigen presenting cells (APC) as feeder cells for NK cells using this technique, it was observed that the expansion system could induce similar NK cell proliferation level compared to standard feeder cell system. Surprisingly, this expansion system not only increased the expression level of NK activation receptors but also enhanced the cytotoxicity of NK cells against tumors.

Accordingly, described herein is a method of expanding NK cells. The method includes providing a population of internally gelated cells, each of which includes a gelated interior and a fluid cell membrane that contains one or more membrane-bound proteins each or collectively are capable of stimulating expansion of NK cells; and culturing a population of cells containing NK cells, which are capable of responding to the one or more membrane-bound proteins, with the population of internally gelated cells under conditions that allow expansion of NK cells. In other words, the internally gelated cells are used as feeder cells.

The population of NK cells can be selected from the group consisting of peripheral blood mononuclear cells (PBMC), NK cells enriched from PBMCs or other sources, iPSC-derived NK cells, embryonic stem cell-derived NK cells, tissue resident NK cells, splenocytes, cord blood cells, and hematopoietic stem cell-derived NK cells.

The one or more membrane-bound proteins can be selected from the group consisting of 41BBL, IL-15, IL-21, B7-H6, BAT3, HLA-DP, HLA-E, HLA-C2, HLA-A, HLA-C, HLA-G, HLA-F, HLA-C, MICA/MICB, ULBP-1, ULBP-2, ULBP-3, ULBP-4, ULBP-5, ULBP-6, AICL, CD48, NTB-A, 2B4, CD2, CD58, CD11a, ICAM1, CRACC, OX40L, CD137L, Nectin-1, Nectin-2, Nectin-3, Nectin-4, necl-1, necl-2, necl-3, necl-4, necl-5, PCNA, AICL, IgG, CD27L, CD72, CEACAM-1, CEACAM-5, OCIL, N-Cadherin, E-Cadherin, R-Cadherin, sialic acid, IL-1, IL-2, IL-4, IL-7, IL-9, IL-12, IL-18, IL-27, IL-33, IL-6, IL-11, CNTF, LIF, OSM, CT-1, CLC, IFN-a, INF-b, CCL-5, and an agonist of TLR-1, TLR-2, TLR-3, TLR-5, TLR-6, TLR-9, NOD-1, NOD-2, NOD-3, or NLRP3. For example, the one or more membrane-bound proteins can include 41BBL and IL-15.

The culturing step can be carried out in a medium suitable for culturing and expansion of NK cells in the presence of IL-21 (e.g., 50 to 200 ng/ml) or IL-2 (e.g., 5 to 200 IU/ml).

In some embodiments, the NK cells and the internally gelated cells can be co-cultured with an NK cell:gelated cell ratio of 1:0.5-20 (e.g., 1:1, 1:2, 1:3, 1:4, 1:5, 1:10, or 1:15) in the presence of IL-21 or IL-2.

The population of internally gelated cells can be generated by a procedure including transiently permeabilizing the lipid membrane of a population of precursor cells in order to introduce an inactive but activable crosslinker into the cells. After the crosslinker enters the permeabilized cells, the cells are allowed to return to their non-permeable state, thus sealing the crosslinker inside the cells. Any remaining extravesicular crosslinker is then removed, for example, by washing the cells. The internal crosslinker is subsequently activated to achieve internal gelation of the cells without disturbing the membrane. The permeabilization step can be carried out in the presence of the crosslinker. Also see WO2018/026644.

The resulting internally gelated cells retain their native exterior and are not susceptible to environmental stresses. Membrane lipids and proteins retain their mobility upon internal gelation. The internally gelated cells produced by the method have a fixed or gelated interior enclosed by a lipid membrane that is substantially identical to the lipid membrane of their precursor cells. Properties of the precursor cells such as sensitivity to a surfactant, membrane fluidity, membrane protein mobility, membrane permeability, membrane content, surface charge, and membrane biological functions can be preserved in the gelated cells.

Various techniques known in the art can be applied to induce transient membrane poration or permeability in the precursor cells. The techniques include, but are not limited to, freeze-and-thaw treatment, osmotic shock, sonoporation, electroporation, laser-induced membrane poration, shear-induced membrane poration, and other techniques based on mechanical means. For example, sonoporation occurs when cavitation events occur in close proximity to a lipid membrane. The interaction between microbubbles and the membrane creates transient pores by acoustic microstreaming, bubble oscillations, shock waves, and microjet formation that puncture the lipid membrane. A skilled person would be able to determine how to apply a technique in order to create temporary pores in the membrane without permanently disrupting the membrane. Typically, upon cessation of the application of a transient membrane poration technique, the generated pores will close spontaneously.

Any crosslinker capable of entering a permeabilized lipid membrane and being activated within cells to create a gelated interior can be utilized to create internally gelated cells. In some embodiments, the crosslinker is a monomer or polymer that can be activated to be crosslinked to form a gel. Thermo-responsive hydrogel crosslinking, photo-responsive hydrogel crosslinking, pH-sensitive hydrogel crosslinking, chemical-responsive hydrogel crosslinking, and sol-gel silica crosslinking are exemplary activable crosslinking techniques.

Photopolymerization or photo-reactive crosslinking is can be used. Photopolymerization is the crosslinking of a polymer that changes its properties upon exposure to light, often in the ultraviolet or visible region of the electromagnetic spectrum, resulting in material curing and hardening. The process can be done in the presence or absence of a photoinitiator. Examples of photoinitiators include, but are not limited to, cationic photoinitiators (e.g., onium salts, organometallic, and pyridinium salts), and free radical photoinitiators (e.g., benzophenone, xanthones, quinones, benzoin ethers, acetophenones, benzoyl oximes, and acylphosphines). Examples of photo-reactive crosslinkers include, but are not limited to, epoxides, urethanes, polyethers, and polyesters of any molecular weight. Photo-reactive crosslinkers are typically functionalized with acrylate for crosslinking. For example, polyethyleneglycol diacrylate (PEGDA) with a molecular weight of 700 can be used with (2-hydroxy-4′-(2-hydroxyethoxy)-2-methylpropiophenone (I-2959) as the photoinitiator.

Thermo-responsive polymers typically contain hydrophobic groups or groups susceptible to chain aggregation at a critical temperature. A thermo-responsive polymer can be introduced into permeablized cells at a specific temperature (i.e., a non-reactive temperature) and subsequently crosslinked by changing the temperature to the critical temperature. Examples of thermo-sensitive polymers applicable to the internal gelation method described herein include, but are not limited to, polyacrylamide derivatives containing hydrophobic pendant groups, PEG-PLGA-PEG triblock copolymers, hydroxyethyl methacrylate-methyl methacrylate (HEMA-MMA), polyacrylonitrile-polyvinyl chloride (PAN-PVC), poly(N-isopropyl acrylamide) (polyNIPAM), poly(N-vinylcaprolactam), cellulose derivatives, ethylene oxide-propylene, and Matrigel.

In any of the methods described herein, internally gelated cells can be generated by providing a population of precursor cells that express one or more membrane-bound proteins. The population of precursor cells is then suspended in phenol-red free DMEM containing a protease inhibitor cocktail to generate a first cell suspension. A gelation solution is added to the first cell suspension to generate a second cell suspension. The gelation solution, which contains a photo-reactive crosslinker and an optional photo-initiator, is capable of increasing membrane permeability of the precursor cells. The second cell suspension is incubated at room temperature for a period of time sufficient to allow the photo-reactive crosslinker and the optional photo-initiator to enter the precursor cells. The second cell suspension is then centrifuged to generate a cell pellet, which is resuspended in phenol-red free DMEM to generate a third cell suspension. A light is applied to the third cell suspension for a period of time sufficient to allow cross-linking of the photo-reactive crosslinker, whereby the population of internally gelated cells is generated. The internally gelated cells are collected and washed. The thus generated internally gelated cells each include a gelated interior and a fluid cell membrane that contains the one or more membrane-bound proteins expressed by the precursor cells.

The gelation solution can be prepared by dissolving a photo-initiator (e.g., I-2959) in dimethyl sulfoxide (DMSO) to create a solution and mixing the solution with a photo-reactive crosslinker (e.g., PEG-DA). In some embodiments, in the gelation solution, I-2959 can range from 0.01 to 1 wt %, and PEG-DA can have an average molecular weight between 200 Da to 5000 Da ranging from 2 to 80 wt %. For example, the gelation solution can be prepared first by dissolving 20 μL of 750 mg/mL of 1-2959 in DMSO and then mixing the resulting solution with 200 μL of PEG-DA.

In some cases, the gelation solution is prepared and added to the first cell suspension such that the second cell suspension has an osmotic concentration of 290 mOsmol to 320 mOsmol, greater than 320 mOsmol or lower than 290 mOsmol.

The gelation solution can be prepared and added to the first cell suspension such that the second cell suspension contains 0.1 to 5 wt % of DMSO.

The stiffness of the gelated cells can be varied by adjusting the concentration of the crosslinker in the second cell suspension. For example, the gelation solution can be added to the first cell suspension such that the concentration of the photo-reactive crosslinker in the second cell suspension is 5 wt % to 50 wt % (e.g., 5 wt %, 10 wt %, 15 wt %, 20 wt %, 25 wt %, 30 wt %, 35 wt %, and 40 wt %).

The expanded NK cells produced by the method described herein can be administered to a subject in need thereof. The expanded NK cells can be derived from a population of cells (e.g., PBMCs) obtained from the subject or another donor subject. The expanded NK cells can be used to treat a cancer, an infection, an autoimmune disorder or NK cell-deficient condition, such as classical NK deficiencies and functional NK deficiencies, or to eradicate unwanted cells.

The internally gelated cells described herein preserve the antigen presenting capability of antigen-presenting cells but lack proliferative activity. The gelated cells thus retain their ability to modulate immune responses without the risk of tumorigenicity. Therefore, the gelated cells can be administered to a subject in need thereof to treat a condition or to induce an immune response. In some cases, the gelated cells are used as vaccines.

The gelated cells can be formulated as a pharmaceutical composition suitable for various routes of administration, e.g., intravenous, intraarticular, conjunctival, intracranial, intraperitoneal, intrapleural, intramuscular, intrathecal, or subcutaneous route of administration. It can contain a pharmaceutically acceptable carrier, e.g., a buffer or excipient, or an adjuvant.

The specific examples below are to be construed as merely illustrative, and not limitative of the remainder of the disclosure in any way whatsoever.

Without further elaboration, it is believed that one skilled in the art can, based on the description herein, utilize the present disclosure to its fullest extent. All publications cited herein are herein incorporated by reference in their entirety.

### Example 1: Gelated Artificial Antigen Presenting Cells Support Proliferation of NK Cells Ex Vivo

Intracellular hydrogelation of the well-established genetically engineered artificial antigen presenting cells (aAPC) was performed for NK expansion. K562 cells were transduced by lentivirus to express 41BBL and membrane bound IL15 (K562-41BBL-mb15 feeder cells). See, Fujisaki et al., Cancer Res. 69, 4010-4017 (2009).

Peripheral blood mononuclear cells (PBMC) from healthy donors were co-cultured with either irradiated K562-41BBL-mb15 feeder cells (GM) or gelated K562-41BBL-mb15 feeder cells (GC) to selectively support NK cell expansion. Given the critical role of IL-21 in NK-cell maturation and proliferation, the expansion of NK cells in response to recursive stimulation by GC and GM with or without IL-21 was compared. As shown in FIG. 3(A), high enrichment of CD3⁻CD56⁺ NK cells (83.5%±10.10%, 89.2%±2.35% for GM, IL-21+GM and 69.0%±22.76%, 93.0%±4.55% for GC, IL-21+GC) after co-culturing with aAPCs for 14 days in all conditions was observed. By day 7, a mean 13.1 fold expansion of NK cells was observed when co-cultured with GC compared to 5.4 fold expansion with GM. Surprisingly, despite the lower number of NK cells when co-cultured with GC, the addition of IL-21 to the GC co-culture showed a significant increase in expansion (12.6 fold for IL-21+GC). By day 14, similar trends of NK expansion by aAPC were observed (66.7±13.0, 75.8±33.5 fold for GM, IL-21+GM and 39.7±9.5, 75.3±6.7 fold for GC, IL-21+GC), suggesting that gelated aAPC kept the ability to support proliferation of NK cells ex vivo and promoted the selective enrichment of expanded NK cells. See FIG. 3(C).

### Example 2: NK Cell Expansion Supported by Gelated aAPC Resulted in Different Expression Patterns of NK Cell Receptors

As GC and GM were derived from the same genetically engineered aAPC, it was expected that NK cells expanded with GC or GM were activated through the same signaling pathway and should have resulted in similar immunophenotypes. To clarify this question, CD3⁻CD56⁺ NK cells were fixed to assess surface expression of the major NK cell receptors before or after expansion by CyTOF. Unexpectedly, as demonstrated in the unsupervised hierarchical clustering analysis, three distinct groups of unexpanded, GC-expanded and GM-expanded groups were clustered together (data not shown). Notably, although both groups expressed higher level of activating receptors than unexpanded NK cells, GC expanded NK cells showed increased expression of activating receptors, including NKp30, CD137, CRACC and NKG2D, as well as perforin, as compared to GM expanded NK cells (data not shown). These results suggested that co-culturing with GC might provide sustained activation signals to NK cells and result in higher expression level of activating receptors and perforin.

### Example 3: Gelated aAPC-Expanded NK Cells Showed Enhanced Cytotoxicity Against Tumor Cell Lines

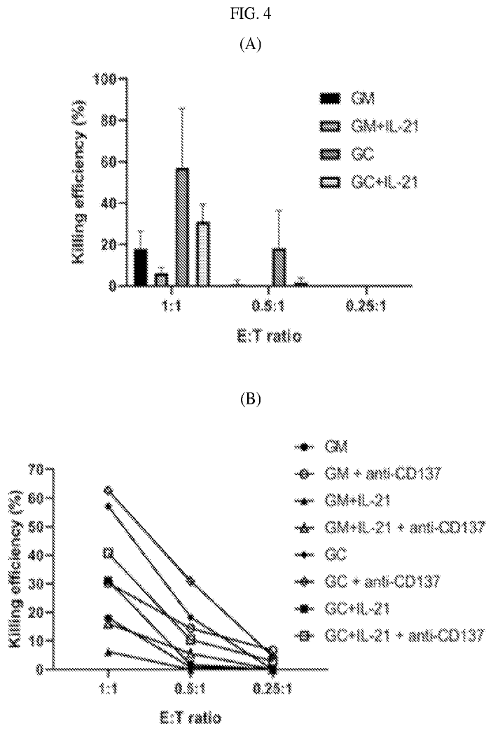

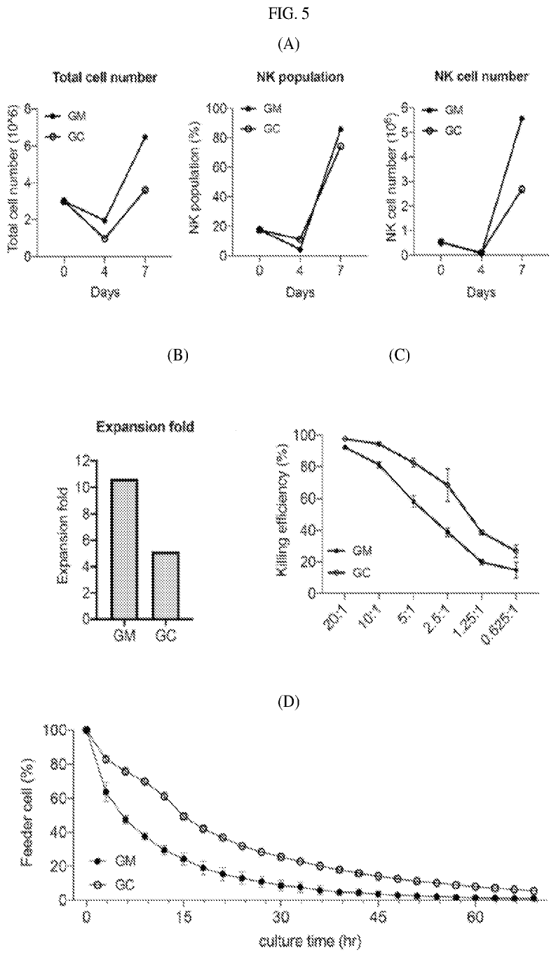

With higher expression of activating receptors and perforin, NK cells expanded with GC could be more cytotoxic against tumor targets. To address this question, the cytotoxicity of expanded NK cells was evaluated by a killing assay. Indeed, it was found that the specific killing of NK cells expanded with GC with or without IL-21 against the target tumor cell line, K562, was much greater than that of GM-expanded NK cells. See FIG. 4(A) In addition, due to higher expression of CD137 in GC-expanded NK cells, GC-expanded NK cells could be further activated by applying anti-CD137 agonist antibodies. As demonstrated in FIG. 4(B), in the presence of anti-CD137 agonist antibody, the cytotoxicity of GC-expanded NK cells against K562 target cells was further enhanced compared to the GM group, suggesting the possibility of combining anti-CD137 agonist with GC-expanded NK cells as a therapeutic strategy.

### Example 4: Gelated aAPC had Higher Persistence and Produced Expanded NK Cells with Greater Cytolytic Activity

To further assess NK cells expanded by gelated feeder cells, gelated cells were generated from K562-41BBL-mb15 cells. PBMC were co-culture with GM or GC. Total cell number, NK population, and NK cell number were determined on day 0, day 4, and day 7. See FIG. 5(A). After expansion for 7 days, NK cells were enriched and analyzed for cytolytic activity. See FIG. 5(C). Although the NK population in both groups were similar on day 7, GM could expand more NK cells than GC group. See FIGS. 5(A) and (B). On the other hand, GC-expanded NK cells exhibited higher cytolytic activity than GM-expanded cells, which suggested that GC had a higher potential to promote NK activity than GM. See FIG. 5(C). It was also observed that GC had higher persistence than GM. See FIG. 5(D). Accordingly, GC might be able to stimulate NK cells for a longer period and induce higher cytolytic activity in NK cells.

### Example 5: Modified Gelated Cells and Culturing Conditions Improved NK Cell Expansion and Cytolytic Activity

To improve GC's ability to expand NK cells, GC properties were modified in order to optimize efficiency.

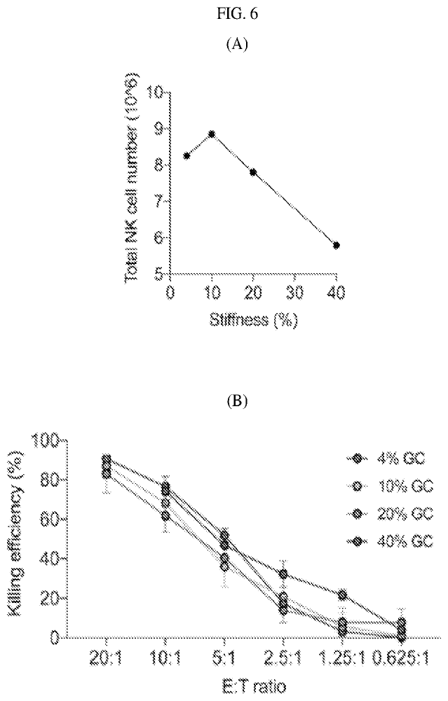

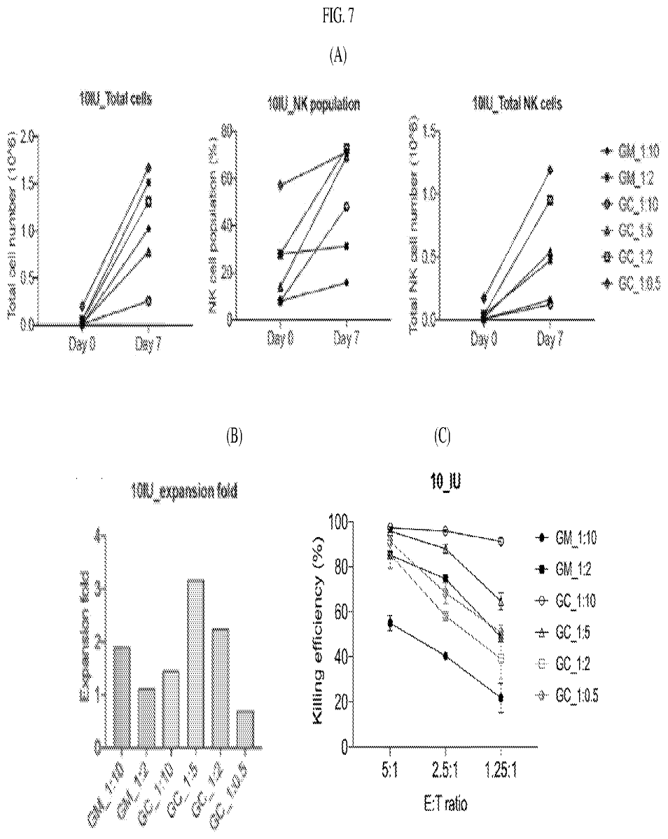

Previous research suggested that the stiffness of the stimulating surface could modulate activation of NK cells. See, Mordechay et al., Mechanical Regulation of the Cytotoxic Activity of Natural Killer Cells (2020), biorxiv.org, doi:10.1101/2020.03.02.972984. The stiffness of GC was adjusted to assess its effect on NK cell activation. GC with different levels of stiffness, including 4%, 10%, 20%, and 40%, were tested. See FIGS. 6(A) and (B). The data showed that the correlation of GC stiffness and NK expansion efficiency was a bell-shape curve, which was consistent with the previous research. 10% stiffness showed the greatest expansion efficiency. See FIG. 6(A). On the other hand, there was no significant difference in cytolytic activity between the different groups of GC. See FIG. 6(B).

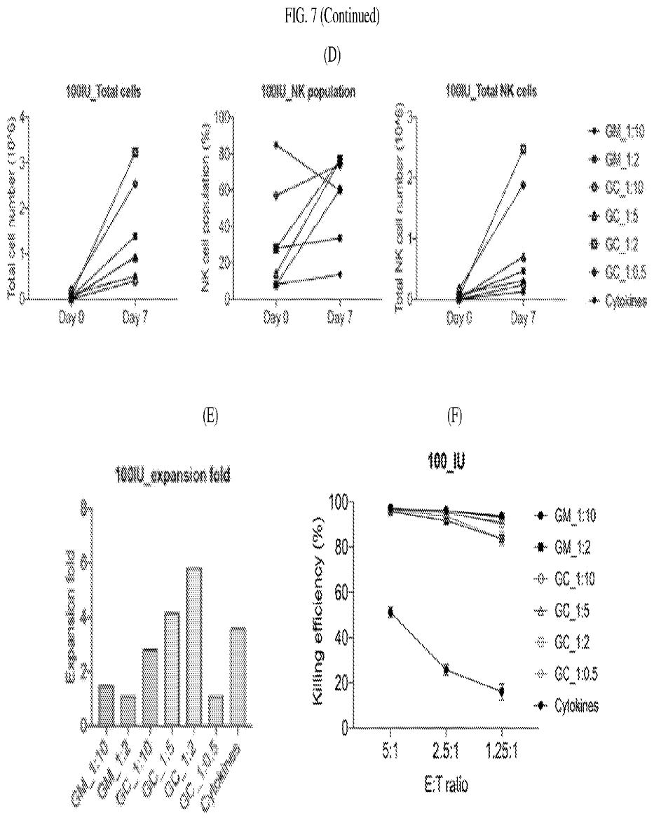

Interaction between NK and other immune cells, such as T cell, could promote NK activation and proliferation. See, Malhotra and Shanker, NK cells immune cross-talk and therapeutic implications. 37 (2012); and Lee et al., Sci Rep 7, 11075 (2017). However, these interactions could impact the quality of NK cells from different batches of expansion. To eliminate this variation, NK cells were enriched from PBMC before expansion. Enriched NK cells were co-cultured with different ratios of NK cells to feeder cells (1:10, 1:5, 1:2, and 1:0.5) in the presence of 10 IU/mL or 100 IU/mL of human IL-2. See FIG. 7. The data showed that NK cells expanded with 100 IU/mL of IL-2 exhibited higher expansion efficiency than cells expanded with 10 IU/mL IL-2. See FIGS. 7(A), (B), (D), and (E). Further, NK cells expanded in the presence of 100 IU/mL IL-2 exhibited higher cytolytic activity. See FIGS. 7(C) and (F). According to these data, NK cells expanded with GC with 10% stiffness, at the NK cells to feeder cells ratio of 1:5, and in the presence of 100 IU/mL IL-2, showed both good expansion efficiency and cytolytic activity. See FIG. 7. Additionally, there was a dramatic difference between GM-expanded and GC-expanded cells at ratio 1:10 with 100 IU/mL of IL-2 on both expansion efficiency and NK cytolytic activity. See FIG. 7(D)-(F).

### Example 6: Materials and Methods

### Cell Lines

K562-41BBL-mb15 cells were a gift from Dr. Chang in NTUH. All cells were cultured in RPMI 1640 media (Gibco) supplemented with fetal calf serum (Hyclone), penicillin (100 U/mL), streptomycin (100 ug/mL).

### Gelated Cells

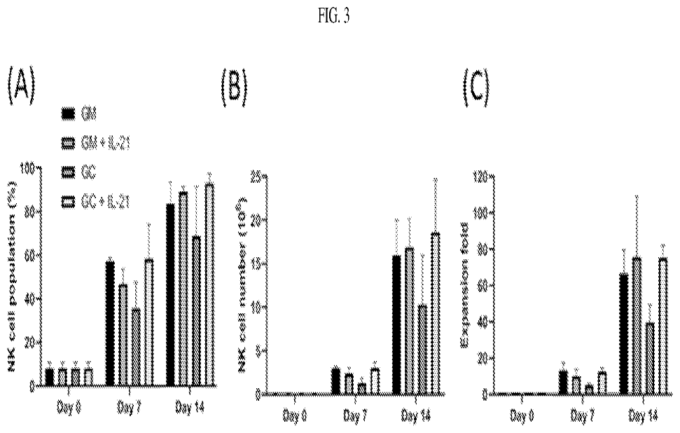

Gelation buffer was first prepared with 20 μL of 750 mg/mL of 2-hydroxy-4′-(2-hydroxyethoxy)-2-methylpropiophenone (Irgacure D-2959; Sigma-Aldrich) dissolved in Dimethyl sulfoxide (DMSO) and mixed with 200 μL of poly(ethylene glycol)-diacrylate (PEG-DA; Mn=700 Da; Sigma-Aldrich). 5×10⁶ K562 cells or genetically modified K562 cells were collected and suspended in 1 mL phenol-red free DMEM (Dulbecco's Modified Eagle Medium) (CA21063-029; ThermoFisher Scientific) containing 1× protease inhibitor. To the cell suspension, the gelation buffer was added at a 1:10 volume ratio to reach a 10 wt % PEG-DA concentration in the cell suspension. Following 5 min of incubation at room temperature, the cells were pelleted and re-suspend in 500 μl pheno-red free DMEM without the gelation buffer and subjected to 365 nm blue-light bombardment for 5 min in an UV oven. The resulting gelated cells (GCs) were washed with PBS once and visually assessed prior to further experimentation. See FIG. 2.

NK Cell Expansion from PBMC

Peripheral blood mononuclear cells (PBMC) were co-cultured with irradiated K562-41BBL-mb15 cells (GMs) or GCs, with or without IL-21 (100 ng/ml) or with or without IL-2 (10 IU/ml or 100 IU/ml) in X-VIVO medium (Lonza) supplemented with 5% human serum (Gemini Bio). To evaluate expansion efficiency, NK population and cell numbers were evaluated after 7 days of expansion. In addition, the expanded NK cells were fixed by paraformaldehyde, and NK activation and inhibition markers were determined by CyTOF. NK activity was assessed by cytotoxicity assay.

### NK Population and Cell Number Determination

PBMC or expanded cells were stained with APC-anti-CD3 (Biolegend) and PE-anti-CD56 (Biolegend), and the NK population (CD3⁻CD56⁺) ratio was validated by flow cytometry. Additionally, the total cell numbers were determined by hemocytometer. NK cell number was calculated as follows: Total cell numbers×NK population ratio.

### Cytotoxicity Assay

NK cell cytotoxic function was assessed by measuring luminance Target cells K562-luc⁺-GFP⁺ stably expressed the luciferase marker. NK cells were co-cultured with target cells at the indicated ratios for 4 hours in triplicate. The cells were lysed, and luminance was determined by the luciferase assay system (promega) in 96 well white plates. Percent cell lysis was calculated as follows: (luminance of target cells alone−luminance of NK-target co-culture)/(luminance of target cells alone−blank)×100%

### Single Cell Mass Cytometry (CyTOF)

Samples were fixed with 1.5% paraformaldehyde at room temperature for 10 min followed by two washes with PBS containing 0.5% BSA. Formaldehyde-fixed cell samples were incubated with metal conjugated antibodies against surface markers for 1 hr, washed once with PBS containing 0.5% BSA, permeabilized with methanol on ice for 10 min, washed twice with PBS containing 0.5% BSA, and then incubated with metal-conjugated antibodies against intracellular molecules for 1 hr. After intracellular staining, cells were washed once with PBS containing 0.5% BSA, and then incubated at room temperature for 10 min with an iridium-containing DNA intercalator (Fluidigm) in PBS containing 1.5% paraformaldehyde. After intercalation/fixation, the cell samples were washed once with PBS containing 0.5% BSA and twice with water before measurement on a CyTOF mass cytometer (Fluidigm). Normalization for detector sensitivity was performed as previously described. After measurement and normalization, individual files were analyzed by first gating out doublets, debris, and dead cells based on cell length, DNA content and cisplatin staining. Heatmap, histogram and ViSNE maps were generated with software tools available at cytobank.org.

### GC Persistence Test

To monitor the persistence of GM and GC in the NK expansion system, a high-content imaging system was used to monitor the GM and GC number change. More specifically, PBMC and feeder cells (GM and GC) were labeled with CellTracker Far Red (Thermo Fisher Scientific) and CFSE (Thermo Fisher Scientific), respectively. PBMC and feeder cells were co-cultured with 10 IU/ml human IL-2 in X-VIVO medium (Lonza) supplemented with 5% human serum (Gemini Bio) for 3 days. Images were acquired every 3 hours by ImageXpress Microsystem (Molecular Devices, Sunnyvale, Calif.) with a 20× objective, FITC and Cy7 filter setup, and 9 fields for each well. Image data were analyzed by ImageJ to evaluate cell number.

### GC Stiffness

GC with different stiffness, including 4%, 10%, 20%, and 40%, were generated by adjusting the concentration of PEG-DA (i.e., 4 wt %, 10 wt %, 20 wt %, and 40 wt %) in the cell suspension containing the gelation buffer. PBMC were co-cultured with the GC with 10 IU/ml human IL-2 in X-VIVO medium (Lonza) supplemented with 5% human serum (Gemini Bio). The culture media was refreshed on day 3 and 5. Total cell number and NK cytolytic function were assessed on Day 7.

### Enriched NK Expansion Test

NK cells were enriched from PBMC by NK isolation kit (Miltenyi Biotec). Then, NK cells were co-cultured with different ratios of NK cells to feeder cells (GM or GC) with 10 or 100 IU/ml human IL-2 in X-VIVO medium (Lonza) supplemented with 5% human serum (Gemini Bio). The media was re-fresh on day 3 and 5. NK cell number, population, and cytolytic function were evaluated on day 7.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

1. A method of expanding natural killer cells, comprising:
providing a population of internally gelated cells, each of which includes a gelated interior and a fluid cell membrane that contains one or more membrane-bound proteins each or collectively are capable of stimulating expansion of natural killer (NK) cells; and
culturing a population of cells containing NK cells, which are capable of responding to the one or more membrane-bound proteins, with the population of internally gelated cells under conditions that allow expansion of NK cells.

2. The method of claim 1, wherein the population of cells is selected from the group consisting of peripheral blood mononuclear cells (PBMC), enriched NK cells, iPSC-derived NK cells, embryonic stem cell-derived NK cells, tissue resident NK cells, splenocytes, cord blood cells, and hematopoietic stem cell-derived NK cells.

3. The method of claim 1, wherein the one or more membrane-bound proteins are selected from the group consisting of 41BBL, IL-15, IL-21, B7-H6, BAT3, HLA-DP, HLA-E, HLA-C2, HLA-A, HLA-C, HLA-G, HLA-F, HLA-C, MICA/MICB, ULBP-1, ULBP-2, ULBP-3, ULBP-4, ULBP-5, ULBP-6, AICL, CD48, NTB-A, 2B4, CD2, CD58, CD11a, ICAM1, CRACC, OX40L, CD137L, Nectin-1, Nectin-2, Nectin-3, Nectin-4, necl-1, necl-2, necl-3, necl-4, necl-5, PCNA, AICL, IgG, CD27L, CD72, CEACAM-1, CEACAM-5, OCIL, N-Cadherin, E-Cadherin, R-Cadherin, sialic acid, IL-1, IL-2, IL-4, IL-7, IL-9, IL-12, IL-18, IL-27, IL-33, IL-6, IL-11, CNTF, LIF, OSM, CT-1, CLC, IFN-a, INF-b, CCL-5, an agonist of TLR-1, TLR-2, TLR-3, TLR-5, TLR-6, TLR-9, NOD-1, NOD-2, NOD-3, and an NLRP3 agonist.

4. The method of claim 2, wherein the one or more membrane-bound proteins include 41BBL and IL-15.

5. The method of claim 1, wherein the culturing step in performed in the presence of IL-21 or IL-2.

6. The method of claim 1, wherein the ratio of the number NK cells to the number of internally gelated cells in the culturing step is 1:0.5-20.

7. The method of claim 1, wherein the population of internally gelated cells is generated by a procedure including:
providing a population of antigen presenting cells that express the one or more membrane-bound proteins;
suspending the population of antigen presenting cells in phenol-red free DMEM containing a protease inhibitor cocktail to generate a first cell suspension;
adding a gelation solution to the first cell suspension to generate a second cell suspension, wherein the gelation solution is capable of increasing membrane permeability of the antigen presenting cells, and contains a photo-reactive crosslinker and an optional photo-initiator;
incubating the second cell suspension at room temperature for a period of time sufficient to allow the photo-reactive crosslinker and the optional photo-initiator to enter the antigen presenting cells;
centrifuging the second cell suspension to generate a cell pellet:
resuspending the cell pellet in phenol-red free DMEM to generate a third cell suspension;
applying a light to the third cell suspension for a period of time sufficient to allow cross-linking of the photo-reactive crosslinker, whereby the population of internally gelated cells is generated; and
collecting and washing the population of internally gelated cells.

8. The method of claim 7, wherein the gelation solution is prepared such that the second cell suspension has an osmotic concentration of 320 mOsmol to 290 mOsmol, greater than 320 mOsmol, or lower than 290 mOsmol.

9. The method of claim 7, wherein the second cell suspension contains 0.1 to 5 wt % of DMSO.

10. The method of claim 7, wherein the concentration of the photo-reactive crosslinker in the second cell suspension is 5 wt % to 50 wt %.

11. The method of claim 7, wherein the photo-reactive crosslinker is poly(ethylene glycol)-diacrylate (PEG-DA), the photo-initiator is 2-hydroxy-4′-(2-hydroxyethoxy)-2-methylpropiophenone, and the light is 365 nm blue light.

12. The method of claim 11, wherein the 2-hydroxy-4′-2(-hydroxyethoxy)-2-methylpropiophenone ranges from 0.01 to 1 wt %, and the PEG-DA has an average molecular weight between 200 Da to 5000 Da ranging from 2 to 80 wt % in the gelation solution.

13. The method of claim 12, wherein the gelation solution is prepared by dissolving 2-hydroxy-4′-(2-hydroxyethoxy)-2-methylpropiophenone in dimethyl sulfoxide (DMSO) to create a solution and mixing the solution with PEG-DA having an average molecular weight of 700 Da.

14. The method of claim 11, wherein the concentration of PEG-DA in the second cell suspension is 10 wt % to 40 wt %.

15. The method of claim 7, wherein the population of antigen presenting cells are artificial antigen presenting cells.

16. The method of claim 15, wherein the artificial antigen presenting cells are or are engineered from K562 cells, PBMC, EBV transformed LCL, 721.221 cells, 8866 cells, Jurkat cells, Jurkat/KL-1 cells, U937 cells, BJAB cells, NB4 cells, 293T cells, MCF7 cells, Jeg3 cells, Hela cells, A549 cells, 1106mel cells, or CEM cells.

17. The method of claim 1, further comprising isolating the expanded NK cells and administering the isolated NK cells to a subject in need thereof.

18. A method of generating a population of internally gelated cells, comprising
providing a population of precursor cells that express one or more membrane-bound proteins;
suspending the population of precursor cells in phenol-red free DMEM containing a protease inhibitor cocktail to generate a first cell suspension;
adding a gelation solution to the first cell suspension to generate a second cell suspension, wherein the gelation solution is capable of increasing membrane permeability of the precursor cells, and contains a photo-reactive crosslinker and a photo-initiator;
incubating the second cell suspension at room temperature for a period of time sufficient to allow the photo-reactive crosslinker and the photo-initiator to enter the precursor cells;
centrifuging the second cell suspension to generate a cell pellet;
resuspending the cell pellet in phenol-red free DMEM to generate a third cell suspension;
applying a light to the third cell suspension for a period of time sufficient to allow cross-linking of the photo-reactive crosslinker, whereby the population of internally gelated cells is generated; and
collecting and washing the population of internally gelated cells;
wherein the internally gelated cells each include a gelated interior and a fluid cell membrane that contains the one or more membrane-bound proteins.

19. The method of claim 18, wherein the gelation solution is prepared such that the second cell suspension has an osmotic concentration of 320 mOsmol to 290 mOsmol, greater than 320 mOsmol, or lower than 290 mOsmol.

20. The method of claim 18, wherein the second cell suspension contains 0.1 to 5 wt % of DMSO.

21. The method of claim 18, wherein the concentration of the photo-reactive crosslinker in the second cell suspension is 5 wt % to 50 wt %.

22. The method of claim 18, wherein the photo-reactive crosslinker is poly(ethylene glycol)-diacrylate (PEG-DA), the photo-initiator is 2-hydroxy-4′-(2-hydroxyethoxy)-2-methylpropiophenone, and the light is 365 nm blue light.

23. The method of claim 22, wherein the 2-hydroxy-4′-2(-hydroxyethoxy)-2-methylpropiophenone ranges from 0.01 to 1 wt %, and the PEG-DA has an average molecular weight between 200 Da to 5000 Da ranging from 2 to 80 wt % in the gelation solution.

24. The method of claim 23, wherein the gelation solution is prepared by dissolving 2-hydroxy-4′-(2-hydroxyethoxy)-2-methylpropiophenone in dimethyl sulfoxide (DMSO) to create a solution and mixing the solution with PEG-DA having an average molecular weight of 700 Da.

25. The method of claim 22, wherein the concentration of PEG-DA in the second cell suspension is 10 wt % to 40 wt %.

26. The method of claim 18, wherein the population of precursor cells are artificial antigen presenting cells.

27. The method of claim 26, wherein the artificial antigen presenting cells are or are engineered from K562 cells, PBMC, EBV transformed LCL, 721.221 cells, 8866 cells, Jurkat cells, Jurkat/KL-1 cells, U937 cells, BJAB cells, NB4 cells, 293T cells, MCF7 cells, Jeg3 cells, Hela cells, A549 cells, 1106mel cells, or CEM cells.

28. The method of claim 26, wherein the one or more membrane-bound proteins each or collectively are capable of stimulating expansion of natural killer (NK) cells.

29. The method of claim 28, wherein the one or more membrane-bound proteins are selected from the group consisting of 41BBL, IL-15, IL-21, B7-H6, BAT3, HLA-DP, HLA-E, HLA-C2, HLA-A, HLA-C, HLA-G, HLA-F, HLA-C, MICA/MICB, ULBP-1, ULBP-2, ULBP-3, ULBP-4, ULBP-5, ULBP-6, AICL, CD48, NTB-A, 2B4, CD2, CD58, CD11a, ICAM1, CRACC, OX40L, CD137L, Nectin-1, Nectin-2, Nectin-3, Nectin-4, necl-1, necl-2, necl-3, necl-4, necl-5, PCNA, AICL, IgG, CD27L, CD72, CEACAM-1, CEACAM-5, OCIL, N-Cadherin, E-Cadherin, R-Cadherin, sialic acid, IL-1, IL-2, IL-4, IL-7, IL-9, IL-12, IL-18, IL-27, IL-33, IL-6, IL-11, CNTF, LIF, OSM, CT-1, CLC, IFN-a, INF-b, CCL-5, an agonist of TLR-1, TLR-2, TLR-3, TLR-5, TLR-6, TLR-9, NOD-1, NOD-2, NOD-3, and an NLRP3 agonist.

30. A population of internally gelated cells generated by the method of claim 18, wherein each cell comprises a gelated interior and a fluid cell membrane that contains the one or more membrane-bound proteins.

31. A composition comprising the population of internally gelated cells of claim 30.

32. A method of inducing an immune response in a subject, comprising administering the composition of claim 31 to the subject.
